(19) United States
(12) Patent Application Publication
(10) Pub. No.: US 2018/0028712 A1
(43) Pub. Date: Feb. 1, 2018

(54) SKIN-REGENERATING MATERIAL COMPRISING SYNERGISTIC COMBINATION OF METAL OXIDES

(71) Applicant: ARGAMAN TECHNOLOGIES LTD., Jerusalem (IL)

(72) Inventor: Mechael KANOVSKY, Rehovot (IL)

(73) Assignee: ARGAMAN TECHNOLOGIES LTD., Jerusalem (IL)

(21) Appl. No.: 15/549,029

(22) PCT Filed: Feb. 7, 2016
(86) PCT No.: PCT/IL2016/050139

§ 371 Date: Aug. 4, 2017

Related U.S. Application Data
(60) Provisional application No. 62/113,450, filed on Feb. 8, 2015.

(51) Int. Cl. A61L 15/44; A61K 33/38; A61K 47/34; A61Q 19/08; A61K 9/70; A61L 15/26; A61L 15/28; A61K 8/19; A61K 33/34; A61K 47/38

(52) U.S. Cl. CPC A61L 15/44; A61K 33/34; A61K 33/38; A61K 47/34; A61K 47/38; A61K 9/70; A61L 15/26; A61L 15/28; A61K 8/19; A61Q 19/08; A61L 2300/104

(57) ABSTRACT

Provided are materials including cell proliferation properties. The materials may include a polymer having incorporated therein a synergistic combination of at least two metal oxide powders, including a mixed oxidation state oxide of a first metal and a single oxidation state oxide of a second metal. The mixed oxidation state oxide may constitute from about 25% wt. to about 75% wt. of the total weight of the synergistic combination of the at least two metal oxide powders. The powders may be incorporated substantially uniformly within the polymer. The ions of the metal powders may be in ionic contact upon exposure of the material to moisture. Further provided are methods for the preparation of the materials and uses thereof, including in skin regeneration processes and cosmetic applications.

### FIELD OF THE INVENTION

The present invention relates to materials comprising a polymer having a synergistic combination of metal oxides incorporated within the polymer, the materials having wound healing and cosmetic properties.

### BACKGROUND OF THE INVENTION

Wound healing is an intricate process where the skin (or another organ-tissue) repairs itself after injury. In normal skin, the epidermis (outermost layer) and dermis (inner or deeper layer) exist in a steady-state equilibrium, forming a protective barrier against the external environment. Once the protective barrier is broken, the normal (physiologic) process of wound healing is immediately set in motion. The classic model of wound healing is divided into three or four sequential, yet overlapping, phases: hemostasis, inflammation, proliferation and remodeling. Upon injury to the skin, a set of complex biochemical events takes place in a closely orchestrated cascade to repair the damage. The physiologic process of wound healing can, however, be compromised in certain conditions, such as, for example in people suffering from diabetic ulcers or from decubiti, commonly known as bed sores; or in immuno-compromised patients.

Certain individual metal oxides are known to stimulate closure of wounds in certain circumstances. It has been observed that metal oxide treated gauze, non-woven material, or yarns in a sleeve, sock or textile have assisted in wound closure. It has been further observed that the wound closure occurs without the creation of fibrous scar tissue using gauze treated with copper oxide. The absence of fibrous scar tissue indicates a stimulation of regenerative mechanisms i.e. cell proliferation rather than, or in addition to, repair mechanisms of the skin (Borkow, Gabbay, et al., Wound Repair and Regeneration, 2010; 18, 266-275). This regeneration is not the case when the body creates scar tissue (Borkow, Levy and Gabbay, Wounds, 2010; 22 (12):301-310). European Patent No. EP1809306 encompasses wound-healing materials which comprise a polymeric fiber, ligament, film, sheet or sheath having embedded directly therein microscopic particles of copper (I) oxide and/or copper (II) oxide in powdered form which release Cu⁺ ions, Cu⁺⁺ ions or combinations thereof upon contact with a fluid, with a portion of said particles being exposed and protruding from surfaces thereof, or comprises a cellulosic fiber plated with the copper oxide, for use in bringing in contact with a body surface having wounds selected from sores, abrasions, ulcerations, lesions, cutaneous openings or burns for the treatment and healing thereof.

It has further been found that copper oxide has skin regeneration properties, when applied to the skin in the form of a cosmetic composition or incorporated into fabrics.

US Patent Application Publication No. 2009/0010969 is directed to a cosmetic method for preventing, minimizing and removing wrinkles and providing for smoother and more robust skin surfaces comprising applying a material incorporating water-insoluble copper compounds which release Cu⁺⁺ ions, Cu⁺⁺ ions or combinations thereof upon contact with a fluid to a body surface to be treated.

US Patent Application Publication No. 2014/0065196 is directed to a composition formulated for topical application for the prevention, mitigation or abrogation of skin aging or skin imperfections in a subject, wherein the composition comprises an insoluble copper oxide as a primary active ingredient therein, wherein said insoluble copper oxide is present at a concentration of between about 0.15%-about 0.75% w/w, the insoluble copper oxide is present as particles ranging in a size of from between about 0.5 to about 10 microns or a combination thereof.

The effect of cell proliferation activities of some single oxidation state metal oxides (such as copper oxide) alone or mixed oxidation state metal oxides (such as tetrasilver tetroxide) alone has been demonstrated in laboratory and in-vitro tests as well as in actual wound healing tests on human tissues including tissue culture studies on: muscle, bone marrow, dorsal root ganglia, and spinal cord neurons. This indicates that the mechanism for cell proliferation using those single or mixed oxidation state metal oxides is common to many cell types. The mechanism for cell proliferation is described in the literature as an angiogenic and epithelial stimulation (Borkow, Levy, Gabbay, Wounds, 2010; 22 (12):301-310.). In the cited reference there is photographic evidence of the effect of copper oxide on reduced scarring in healing wounds. Similar findings have also been published in connection to tetrasilver tetroxide as a mixed oxidation state compound as cited in various publications and patents by Antelman.

U.S. Pat. No. 6,258,385 to Antelman is directed to methods for treating dermatological skin disease comprising applying a composition comprising a therapeutically effective amount of tetrasilver tetroxide directly to the affected skin of a patient in need of treatment, said composition being free of added oxidizing agent, wherein the dermatological skin disease is selected from the group consisting of eczema, psoriasis, dermatitis, ulcers, shingles, rashes, bedsores, cold sores, blisters, boils, herpes, acne, pimples, warts, and a combination thereof.

U.S. Pat. No. 6,669,966 to Antelman discloses skin-growth-enhancing compounds and compositions including a therapeutically effective amount of at least one electron active compound, or a pharmaceutically acceptable derivative thereof, that has at least two polyvalent cations, at least one of which has a first valence state and at least one of which has a second, different valence state. Preferred compounds include Bi(III,V) oxide, Co(II,III) oxide, Cu(I,III) oxide, Fe(II,III) oxide, Mn(II,III) oxide, and Pr(III,IV) oxide, and Ag(I,III) oxide, or a combination thereof. These compounds may be in a crystalline state having metallic cations of two different valences, or electronic states, in the inorganic crystal.

U.S. Pat. No. 6,645,531 to Antelman discloses pharmaceutical compositions that include a therapeutically effective amount of at least one electron active compound, or a pharmaceutically acceptable derivative thereof, that has at least two polyvalent cations, at least one of which has a first valence state and at least one of which has a second, different valence state. Preferred compounds include Bi(III,V) oxide, Co(II,III) oxide, Cu(I,III) oxide, Fe(II,III) oxide, Mn(II,III) oxide, and Pr(III,IV) oxide, and optionally Ag(I,III) oxide. These compounds may be in a crystalline state having metallic cations of two different valences, or electronic states, in the inorganic crystal.

There is an unmet need for a cost-effective material having improved cell proliferation properties, which can be beneficially used in dermal applications, such as wound healing and skin regeneration.

### SUMMARY OF THE INVENTION

The present invention relates to skin-regenerating materials having cell proliferation properties and methods for the preparation thereof. The skin-regenerating material comprises a polymer and a synergistic combination of at least two metal oxide powders incorporated within the polymer, comprising a mixed oxidation state oxide and a single oxidation state oxide. The metal oxide powders are incorporated within the polymer such that upon exposure of the material to moisture, the ions of the two metal oxides are in ionic contact with each other.

The present invention is based in part on an unexpected discovery that the cell proliferation activity of a single oxidation state metal oxide is enhanced by the addition of a mixed oxidation state metal oxide, wherein the two metal ions are in ionic contact, such that the combination of the metal oxide particles provides a synergistic effect as compared to the activity of each of the metal oxides alone. It has further been surprisingly found that the cell proliferation activity of the synergistic combination increased when the mixed oxidation state oxide and the single oxidation state oxide were present in the combination in a substantially equal amount.

Homogeneous incorporation of inorganic particles into a substrate, particularly a polymeric substrate, is challenged by particle agglomeration, chemical and physical interaction between the particles and the substrate and most of all by difference in the specific gravities of the particulate materials. However, materials of the present invention, which in some embodiments comprise particulate metal oxides having substantially different specific gravities are generally characterized by a homogeneous distribution of the metal oxide powders within the polymer material. The present invention overcomes the problem imposed by use of distinct types of metal oxides by equalizing bulk densities of the metal oxide particles. Thus, according to some embodiments, the materials of the present invention comprise metal oxide powders, which, even though having substantially different specific gravities, have substantially similar bulk densities. Mean particle sizes of the metal oxides can be proportionally reduced in order to compensate for the difference in the specific gravities thereof and obtain substantially similar bulk densities. Alternatively, bulk densities of the metal oxides can be equalized by coating the metal oxide powders with a coating, which thickness or weight is proportional to the specific gravity of the powders.

Therefore, according to one aspect, the present invention provides a material having cell proliferation properties, said material comprising a polymer having incorporated therein synergistic combination of at least two metal oxide powders comprising a mixed oxidation state oxide of a first metal and a single oxidation state oxide of a second metal, the powders being incorporated substantially uniformly within said polymer, wherein the mixed oxidation state oxide constitutes from about 25% wt. to about 75% wt. of the total weight of the synergistic combination of the at least two metal oxide powders, and wherein the ions of the metal oxides are in ionic contact upon exposure of said material to moisture.

According to some embodiments, the at least two metal oxide powders have substantially different specific gravities. In further embodiments, the at least two metal oxide powders have substantially similar bulk densities.

According to some embodiments, the first metal and the second metal are different.

In some embodiments, the mixed oxidation state oxide is selected from the group consisting of tetrasilver tetroxide (Ag₄O₄), Ag₃O₄, Ag₂O₂, tetracopper tetroxide (Cu₄O₄), Cu (I,III) oxide, Cu (II,III) oxide, Cu₄O₃ and combinations thereof. Each possibility represents a separate embodiment of the invention. In certain embodiments, the mixed oxidation state oxide is selected from the group consisting of tetrasilver tetroxide (Ag₄O₄), Ag₂O₂, tetracopper tetroxide (Cu₄O₄), Cu (I,III) oxide, Cu (II,III) oxide and combinations thereof. Each possibility represents a separate embodiment of the invention.

In some embodiments, the single oxidation state oxide is selected from the group consisting of copper oxide, silver oxide, zinc oxide and combinations thereof. Each possibility represents a separate embodiment of the invention. Copper oxide may be selected from the group consisting of cuprous oxide (Cu₂O), cupric oxide (CuO) and combinations thereof. Each possibility represents a separate embodiment of the invention.

In particular embodiments, the combination of the at least two metal oxides comprises copper oxide and tetrasilver tetroxide. In further particular embodiments, copper oxide is cuprous oxide. According to further embodiments, the mixed oxidation state oxide constitutes from about 40% wt. to about 60% wt. of the total weight of the synergistic combination of the at least two metal oxide powders.

According to some embodiments, the single oxidation state oxide constitutes from about 25% wt. to about 75% wt. of the total weight of the synergistic combination of the at least two metal oxide powders. According to further embodiments, the single oxidation state oxide constitutes from about 40% wt. to about 60% wt. of the total weight of the synergistic combination of the at least two metal oxide powders.

According to certain embodiments, the mixed oxidation state oxide and the single oxidation state oxide are each present in the synergistic combination in a weight percent of about 50%. According to some embodiments, the metal oxide powders are not exposed on the surface of the material. According to other embodiments, the powders are distributed on the surface of the material in a generally uniform fashion.

According to some embodiments, the metal oxide powders comprise metal oxide particles. According to further embodiments, the metal oxide particles protrude from a surface of the material. In yet further embodiments, the metal oxide particles are attached to, deposited on or inserted into the surface of the material.

According to some embodiments, each of the metal oxide powders independently comprises particles, having a mean particle size of from about 1 nanometer to about 10 microns. According to other embodiments, each of the metal oxide powders independently comprises particles, which size is from about 10 nanometers to about 10 microns. According to further embodiments, each of the metal oxide powders independently comprises particles, which size is from about 0.5 to about 1.5 microns.

According to some embodiments, the metal oxide powders having the substantially similar bulk densities comprise particles which mean particle size is inversely proportional to the specific gravity thereof. According to other embodiments, the metal oxide powders having the substantially similar bulk densities comprise particles which have substantially similar mean particles sizes and wherein said particles comprise a coating. According to further embodiments, the coating thickness is proportional to the specific gravity of the metal oxide particles. In alternative embodiments, the coating weight is proportional to the specific gravity of the metal oxide powders. According to further embodiments, the coating comprises polyester or polyalkene wax. The polyester or polyalkene wax may be selected from the group consisting of a polypropylene wax, oxidized polyethylene wax, ethylene homopolymer wax and a combination thereof. Each possibility represents a separate embodiment of the invention.

According to further embodiments, the metal oxide powders comprise particles, which are encapsulated within an encapsulating compound. The encapsulating compound may comprise silicate, acrylate, cellulose, derivatives thereof or combinations thereof. The non-limiting example of acrylate is poly(methyl methacrylate) (PMMA). According to the some exemplary embodiments, the encapsulating agent is a silicate or a poly(methyl methacrylate) (PMMA).

According to some embodiments, the material of the present invention further comprises a chelating agent or a metal deactivating agent associated with the metal oxide powders. Each possibility represents a separate embodiment of the invention. The metal deactivating agent may be selected from the group consisting of phenolic antioxidant, potassium iodide, potassium bromide, calcium stearate, zinc stearate, aluminum stearate, tertiary chain extender and a combination thereof. Each possibility represents a separate embodiment of the invention.

According to further embodiments, the material of the present invention further comprises a surfactant associated with the metal oxide powder. The surfactant may include a sulfate, a sulfonate, a silicone, a silane, or a non-ionic surfactant. The non-limiting examples of commercially available surfactants include Sigma Aldrich Niaproof®, Dow Corning Xiameter® and Triton-X-100. The surfactant may further comprise a solvent, such as but not limited to, methyl alcohol, methyl ethyl ketone, or toluene. According to some embodiments, the material is devoid of the surfactant.

According to some embodiments, the material of the present invention comprises a polymer selected from a synthetic polymer, naturally occurring polymer or combinations thereof. Each possibility represents a separate embodiment of the invention. According to some embodiments, the synthetic polymer is selected from the group consisting of organic polymers, inorganic polymers and bioplastics. In further embodiments, the polymer is selected from the group consisting of polyamide, polyester, acrylic, polyalkene, polysiloxane, nitrile, polyvinyl acetate, starch-based polymer, cellulose-based polymer, derivatives, dispersions and mixtures thereof. Each possibility represents a separate embodiment of the invention. According to some currently preferred embodiments, the polymer is selected from polyester, polyalkene and polyamide. The polyalkene may be selected from the group consisting of polypropylene, polyethylene and combinations thereof. Each possibility represents a separate embodiment of the invention. According to particular embodiments, the polymer is selected from polyethylene, polypropylene, acrylonitrile butadiene styrene (ABS), polylactic acid (PLA), polyglycolic acid (PGA), poly(lactic-co-glycolic acid) (PLGA), poly(butyl acrylate) (PBA), polybutylene terephthalate (PBT) and combinations thereof. The polymer dispersion may be water based or solvent based. According to some embodiments, the material of the present invention is selected from an intermediate-product, a semi-final product or a final product. In some embodiments, the metal oxide powders are incorporated into the polymer by means of a master batch manufacturing process. Thus, according to some embodiments, the intermediate product is a master-batch. According to some embodiments, the semi-final product comprises a fiber, a yarn, a textile, a fabric, a film or a foil. Each possibility represents a separate embodiment of the invention. The textile can be selected from a woven textile, a knit textile, a non-woven textile, a needle-punch textile or felt. Each possibility represents a separate embodiment of the invention. According to certain embodiments, the semi-final product is a fiber. The fiber can be a filament fiber or a staple fiber. According to some embodiments, the metal oxide powders are incorporated substantially uniformly within the fiber. The fiber can be formed into a yarn, textile or fabric. According to some embodiments, the final product is a textile product or a non-textile polymeric article. According to some embodiments, the yarn, textile or fabric are formed into the textile product.

According to further embodiments, the polymer is formed into a semi-final product or a final product by means of extrusion, molding, casting or 3D printing. Each possibility represents a separate embodiment of the invention. According to some embodiments, the textile product comprises an extruded polymer. According to further embodiments, the semi-final product comprises an extruded polymer. According to certain embodiments, the fiber comprises an extruded polymer. According to additional embodiments, the non-textile polymeric article comprises an extruded, molded or cast polymer.

In some embodiments, the combined weight of the at least two metal oxides constitutes from about 0.25% to about 50% wt. of the total weight of the material.

In some embodiments, the material is in a form of a master batch. In further embodiments, the combined weight of the at least two metal oxides constitutes from about 0.5% to about 50% wt. of the total weight of the master batch. In yet further embodiments, the combined weight of the at least two metal oxides constitutes from about 20% to about 40% wt. of the total weight of the master batch.

In some embodiments, the material is in a form of a fiber, a yarn, a textile or a fabric. In certain such embodiments, the combined weight of the at least two metal oxides constitutes from about 0.5% to about 15% wt. of the total weight of the material.

According to some embodiments, the fiber is a polymeric fiber, being synthetic or semi-synthetic. According to some embodiments, the material further comprises a natural fiber. Thus, in some embodiments, the fiber is a blend of the polymeric fiber with a natural fiber. According to further embodiments, the material comprises natural fiber is a weight percent of up to about 85% of the total weight of the material. In particular embodiments, the material comprises natural fiber is a weight percent of up to about 70% of the total weight of the textile product. The natural fiber may be selected from the group consisting of cotton, silk, wool, linen and combinations thereof. Each possibility represents a separate embodiment of the invention. In a certain embodiment, the material comprises cotton.

According to some embodiments, the material comprises the blend of a polymeric fiber and a natural fiber. In certain such embodiments, the combined weight of the at least two metal oxides constitutes from about 0.25% to about 5% wt. of the total weight of the material.

According to some embodiments, the material is in a form of a textile product or a non-textile polymeric article. Each possibility represents a separate embodiment of the invention. The textile product may be selected from clothing items, bedding textiles, medical textiles including bandages or sutures and textiles for internal and external use. The non-limiting examples of the textile products include pillowcases, eye-masks, gloves, socks, stockings, sleeves, undergarments, sheets, bedding, gauze pads, trans-dermal patches, bandages, adhesive bandages, sutures, sheaths, compression garments in all sizes for different parts of the body, and absorbent pads and textiles for internal use. The non-textile polymeric article may include bandages or sutures, mono-lithic films, breathable films, or absorbent pads.

According to some embodiments, the material is for use in enhancing mammalian cell proliferation. In further embodiments, the material is for use in skin regeneration processes, selected from the group consisting of wound healing, accelerated wound closure, and wound healing with reduced scarring. Each possibility represents a separate embodiment of the invention. The wound can be a naturally caused wound or a surgically-induced wound. In further embodiments, the wound is cutaneous or subcutaneous.

According to yet further embodiments, the material is for use in cosmetic applications, selected from the group consisting of reduction of wrinkles, reduction of small skin defects, reduction of erythema, reduction of edema, softening of skin and reduction of odor.

In another aspect, the present invention provides a method for the preparation of a skin-regenerating material having cell proliferation properties, said material comprising a polymer having incorporated therein a synergistic combination of at least two metal oxide powders, comprising a mixed oxidation state oxide of a first metal and a single oxidation state oxide of a second metal, the powders being incorporated substantially uniformly within said polymer, and wherein the ions of the metal oxides are in ionic contact upon exposure of said material to moisture, the method comprising mixing the at least two metal oxide powders with at least one polymer, wherein the mixed oxidation state oxide constitutes from about 25% wt. to about 75% wt. of the total weight of the synergistic combination of the at least two metal oxide powders. In some embodiments, the at least two metal oxide powders have substantially different specific gravities, In further embodiments, the method comprises processing the at least two metal oxide powders to have substantially similar bulk densities prior to mixing thereof with the polymer. According to some embodiments, the method comprises processing the metal oxide powders to obtain particles having mean particles sizes which are inversely proportional to the specific gravity thereof. In some embodiments, said processing comprises grinding. Additionally or alternatively, the method can comprise processing the metal oxide powders to obtain particles having substantially similar sizes. In some embodiments, said processing comprises grinding. In additional embodiments, said processing of the at least two metal oxide powders to have substantially similar bulk densities further comprises applying a coating to the metal oxide powder particles. In some embodiments, said processing comprises applying a coating to the particles of at least one of the metal oxide powders. In further embodiments, said processing comprises applying a coating to the particles of each of the at least two metal oxide powders. In further embodiments, the coating thickness is proportional to the specific gravity of the metal oxide powders.

In some embodiments, the method further comprises a step of encapsulating the metal oxide powder particles within an encapsulating compound. In other embodiments, the method comprises a step of mixing the metal oxide powders with a metal deactivating agent or a chelating agent. In further embodiments, the method comprises a step of mixing the metal oxide powders with a surfactant.

According to further embodiments, said mixing of the at least two metal oxide powders with at least one polymer comprises producing a master batch, comprising the metal oxide powders and a carrier polymer. According to some embodiments, said at least one polymer comprises the carrier polymer. According to the preferred embodiments, the master batch is homogeneous. The master batch may be formed into pellets. Alternatively, the master batch may be formed into granules.

In some embodiments said mixing further comprises adding the master batch to a polymer slurry. In further embodiments the polymer slurry comprises a polymer, which is the same as the carrier polymer. In other embodiments, the polymer slurry comprises a polymer, which is chemically compatible with the carrier polymer.

According to some embodiments, the method further comprises forming from the obtained mixture a semi-final product selected from a film, a foil, a fiber, a yarn, a fiber or a textile. Each possibility represents a separate embodiment of the invention. The method can further comprise blending the obtained fiber with a natural fiber. In some embodiments, the method comprises forming the fiber into yarn, textile, fabric or a final textile product.

According to additional or alternative embodiments, the method further comprises forming from the obtained mixture a final product selected from a textile product or a non-textile polymeric article. Each possibility represents a separate embodiment of the invention.

In some embodiments, the step of forming a semi-final or a final product comprises extrusion, molding, casting or 3D printing. Each possibility represents a separate embodiment of the invention. In some exemplary embodiments said step comprises extrusion. In further embodiments, extrusion comprises spinning through a spinneret. In the preferred embodiments, the material is homogeneously extruded. In other embodiments said step comprises molding.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1A: SEM micrograph of a polyester staple fiber containing copper oxide and tetrasilver tetroxide, at 200× magnification with protruding particles.

FIG. 1B: SEM micrograph of a polyester staple fiber containing copper oxide and tetrasilver tetroxide, at 1000× magnification with protruding particles.

FIGS. 2A-2C: Photographs of various stages of wound healing, the wound being untreated (FIG. 2A) and treated (FIGS. 2B and 2C, being two separate tests) with polymeric fabrics comprising copper oxide and tetrasilver tetroxide.

### DETAILED DESCRIPTION

The present invention relates to materials having improved cell proliferation properties and to methods for preparation of said materials. Cell proliferation properties can include skin regeneration. The skin-regenerating materials of the present invention comprise a polymer and a synergistic combination of at least two metal oxide powders homogeneously incorporated into said polymer.

As used herein, the terms “cell proliferation”, “cell proliferating” or “cell regeneration”, which can be used interchangeably, refer to an ability of the material to enhance cell proliferation, promoting wound healing or closure; wound healing or closure with reduced scarring, accelerated wound healing, more rapid and complete growth of specialized (non-fibroblast) cells within the wound, improved cosmetic appearance of the wound, greater symptomatic relief; whether the wound was caused by trauma or surgical means, and whether the wound is cutaneous or subcutaneous; angiogenesis; epithelization; improvement to the skin, including, but not limited to regeneration of hair follicles or of sebaceous glands, reduction of wrinkles or of small skin defects, softening of skin, reduction of erythema or of edema or an overall improvement in the appearance of the skin; or regeneration of bone marrow cells, muscle cells, dorsal root ganglia, spinal cord neurons or skin tissue. Each possibility represents a separate embodiment of the invention.

According to some embodiments, the material of the present invention is selected from an intermediate-product, such as, but not limited to, a master batch; a semi-final product, for example, a fiber, a yarn, a textile, a fabric, a film or a foil; or a final product, including, inter alia, a textile product or a non-textile polymeric article.

The synergistic combination of the at least two metal oxide powders comprises a mixed oxidation state oxide of a first metal and a single oxidation state oxide of a second metal, and wherein the ions of the metal oxides are in an ionic contact upon hydration of said material or its exposure to residual moisture. In some currently preferred embodiments, the mixed oxidation state oxide constitutes from about 25% wt. to about 75% wt. of the total weight of the synergistic combination of the at least two metal oxide powders,

As used herein, the term “ionic contact” refers to the ability of ions of each of the metal oxide powders, being incorporated within the polymer, to flow to a mutual aqueous reservoir upon exposure to said reservoir.

### The Synergistic Combination of Two Metal Oxides

It has been surprisingly found that in order to improve cell proliferation properties of a single oxidation state metal oxide, a mixed oxidation state metal oxide compound should be added to the single oxidation state oxide. Without wishing to being bound by theory or mechanism of action, in order to provide the induced cell proliferation activity, the metal oxide particles should be mixed together in such a manner that the particles of each oxide are exposed to the same moisture reservoir, thus enabling a diffusion of ions from each metal oxide compound to the mutual moisture reservoir.

The synergistic combination of the two metal oxides, wherein at least one of the metal oxides is a mixed oxidation state oxide and at least one of the metal oxides is a single oxidation state oxide is a non-naturally occurring biologically active combination. According to some embodiments, said non-naturally occurring combination of metal oxides applied to a polymer substrate demonstrates greater ionic activity than the naturally occurring compounds alone. Without wishing to being bound by theory or mechanism of action, the increased ionic activity is responsible for a greater cell proliferation effect when compared to the equal amounts of naturally occurring metal oxide compounds under similar conditions.

As defined herein, the term “synergistic combination” refers to a combination of at least two metal oxides, which provides higher cell proliferation efficiency than the equal amount of each of the metal oxides alone. The higher cell proliferation efficiency may relate to accelerated wound healing and/or skin regeneration processes.

The synergistic combination applied to a polymer comprises two or more biologically active relatively insoluble metal oxides, wherein at least one metal oxide is selected from single oxidation state oxide compounds, and at least one metal oxide is selected from mixed oxidation state oxide compounds. Said combination of metal oxides has been found to be biologically active by itself and synergistic, providing surprisingly accelerated cell proliferation as compared to the same single and mixed oxidation state metal oxides individually, or to the same single oxidation state metal oxide combined within a different single oxidation state metal oxide, which combinations are naturally occurring.

As used herein, the term “mixed oxidation state” refers to atoms, ions or molecules in which the electrons are to some extent delocalized via various electronic transition mechanisms and are shared amongst the atoms, creating a conjugated bond which affects the physiochemical properties of the material. In the mixed oxidation state, electronic transitions form a superposition between two single oxidation states. This can be expressed as any metal that has more than a single oxidation state coexisting, as in the formula X (Y, Z), where X is the metal element and Y and Z are the oxidation states, where Y≠W. The mixed oxidation state oxide may be one compound, wherein metal ions are in different oxidation states (i.e. X(Y,Z)).

According to some embodiments, the mixed oxidation state oxide useful in the materials of the present invention is selected from the group consisting of tetrasilver tetroxide (TST)—Ag₄O₄ (Ag I, III), Ag₃O₄, Ag₂O₂, tetracopper tetroxide—Cu₄O₄ (Cu I, III), Cu₄O₃, Cu (I, II), Cu (II, III), Co(II,III), Pr(III,IV), Bi(III,V), Fe(II,III), and Mn(II,III) oxides and combinations thereof. Each possibility represents a separate embodiment of the invention. In certain embodiments, the material comprises a mixed oxidation state oxide selected from the group consisting of tetrasilver tetroxide, tetracopper tetroxide and a combination thereof.

As used herein, the term “single oxidation state” refers to atoms, ions or molecules in which same types of atoms are present in one oxidation state only. For example, in copper (I) oxide copper all ions are in the oxidation state +1, in copper (II) oxide all copper ions are in the oxidation state +2 and in zinc oxide all zinc ions are in oxidation state +2.

According to some embodiments, the single oxidation state oxide useful in the materials of the present invention is selected from the group consisting of copper oxide, silver oxide, zinc oxide and combinations thereof.

As used herein, the term “copper oxide” refers to either or both of copper oxide's multiple oxidation states: the first, principal single oxidation state cuprous oxide ((Cu₂O), also identified as copper (I) oxide); or the second, higher single oxidation state cupric oxide ((CuO), also identified as copper (II) oxide) either individually or in varying proportions of the two naturally occurring oxidation states.

As used herein, the term “silver oxide” refers to silver oxide's multiple oxidation states: the first, principal single oxidation state Ag₂O (also identified as silver (I) oxide); or the second, higher single oxidation state AgO, (also identified as silver (II) oxide); or the third highest single oxidation state Ag₂O₃, individually or in any varying proportion of these three naturally occurring oxidation states.

As used herein, the term “zinc oxide” refers to zinc oxide's principal oxidation state ZnO₂. According to some embodiments, copper oxide is selected from the group consisting of Cu₂O, CuO and combinations thereof. According to further embodiments, silver oxide is selected from the group consisting of Ag₂O, AgO, Ag₂O₃ and combinations thereof. Each possibility represents a separate embodiment of the invention.

In certain embodiments, the material comprises a single oxidation state oxide selected from the group consisting of copper oxide, silver oxide and a combination thereof. In further embodiments, the single oxidation state oxide is copper oxide. In still further embodiments, the material comprises a single oxidation state oxide selected from the group consisting of Cu₂O, CuO and combinations thereof. Each possibility represents a separate embodiment of the invention. In certain embodiments, copper oxide is Cu₂O.

According to some embodiments, the metal oxides useful in the materials of the present invention are selected from the group consisting of copper oxide, tetracopper tetroxide, silver oxide, tetrasilver tetroxide, zinc oxide and combinations thereof. According to further embodiments, the metal oxides are selected from the group consisting of Cu₂O, CuO, Cu₄O₄, Ag₂O, AgO, Ag₂O₃, Ag₄O₄, ZnO₂ and combinations thereof. In particular embodiments, the material comprises at least two metal oxides selected from the group consisting of copper oxide, tetrasilver tetroxide, tetracopper tetroxide and combinations thereof. In the currently preferred embodiments, the single oxidation state oxide is copper oxide and the mixed oxidation state oxide is tetrasilver tetroxide. In further embodiments, the single oxidation state oxide is cuprous oxide and the mixed oxidation state oxide is tetrasilver tetroxide.

While acceleration of the skin-regenerating effects of a naturally occurring copper oxide comprising a mixture of cupric and cuprous oxides was disclosed, for example, in US Patent Application US2009/0010969, the present invention provides for the first time non-naturally occurring combinations of metal oxides, specifically combinations comprising a single oxidation state oxide combined with tetracopper tetroxide or tetrasilver tetroxide, such combinations being characterized by synergistic cell proliferation properties. The synergistic effect of the compositions comprising a mixture of two different metal oxides, wherein at least one of the metal oxides is a mixed oxidation state oxide is even more surprising considering cytotoxicity of tetrasilver tetroxide, which was evidenced by the inventor of the present invention, as disclosed hereinbelow. Thus, said cell proliferation activity of the combination of the metal oxides was increased also as compared to the activity of the mixed oxidation state metal alone. Without wishing to being bound by theory or mechanism of action, the measured synergistic effect of such combinations can be attributed to intervalence charge transfer between the metal ions having different oxidation states. Exposure of the combination of the at least two metal oxides, comprising a mixed oxidation state oxide and a single oxidation state oxide, to a mutual moisture reservoir establishes ionic contact between the metal oxides and allows ion release from each metal oxide to the mutual moisture reservoir, thus providing acceleration of the cell proliferation activity.

It has been further surprisingly found that the highest cell proliferation activity was achieved when the mixed oxidation state oxide and the single oxidation state oxide were present in the synergistic combination in substantially equal amounts. Thus, according to some embodiments, the mixed oxidation state oxide constitutes from about 25% wt. to about 75% wt. of the total weight of the synergistic combination of the at least two metal oxide powders, from about 30% wt. to about 70% wt., from about 35% wt. to about 65% wt., from about 40% wt. to about 60% wt., or from about 55% wt. to about 65% wt. of the total weight of the synergistic combination of the at least two metal oxide powders. Each possibility represents a separate embodiment of the invention.

According to some embodiments, the single oxidation state oxide constitutes from about 25% wt. to about 75% wt. of the total weight of the synergistic combination of the at least two metal oxide powders, from about 30% wt. to about 70% wt., from about 35% wt. to about 65% wt., from about 40% wt. to about 60% wt., or from about 55% wt. to about 65% wt. of the total weight of the synergistic combination of the at least two metal oxide powders. Each possibility represents a separate embodiment of the invention.

According to certain embodiments, the mixed oxidation state oxide and the single oxidation state oxide are each present in the synergistic combination in a weight percent of about 50%.

### The Metal Oxide Powders

The copper oxide useful in the materials of the present invention can be any commercially available copper oxide powder with a purity level of no less than 97% wt. In some exemplary embodiments, the powder is purchased from SCM Inc. of North Carolina, USA. Due to the prevalence of suppliers of this powder it is not economically viable to manufacture the powder. The zinc oxide useful in the materials of the present invention can be any commercially available zinc oxide powder with a recommended purity level of no less than 98% wt. which is readily available commercially. However, due to the difficulty in obtaining tetrasilver tetroxide and/or tetracopper tetroxide, it is necessary to synthesize the specific species as described hereinbelow. According to some embodiments, the particle size of the commercially available metal oxide powder is from about 10 to about 20 micron. The metal oxide powder can be ground to a particle size of from about 1 nanometer to about 10 micron. Accordingly, the size of the metal oxide particles in the materials of the present invention can be from about 1 nanometer to about 10 microns. According to some embodiments, the particle size is from about 1 to 10 micron. According to further embodiments, the particle size is from about 5 to about 8 micron. According to other further embodiments, the particle size is from about 0.1 to about 0.5 micron. According to further embodiments, the particle size is from about 0.25 to about 0.35 micron According to some embodiments, the metal oxide powders comprise agglomerates which are no larger than 20 microns. According to other embodiments, the metal oxide powders comprise agglomerates, which are no larger than 10 microns. In other embodiments, the materials of the present invention are devoid of metal oxide particles agglomerates.

### The Polymer

As used herein, the term “polymer” or “polymeric” refers to materials consisting of repeated building blocks called monomers. The polymer may be homogenous or heterogeneous in its form; hydrophilic or hydrophobic; natural, synthetic, mixed synthetic or bioplastic. The non-limiting examples of polymers suitable for incorporation of the metal oxide powders include, inter alia, polyamide, polyester, acrylic, isotactic compounds including but not limited to polypropylene, polyethylene, polyolefin, acrylic compounds, polyalkene, silicones, and nitrile; cellulose-based polymer or a mixture of different cellulose materials; converted cellulose mixed with plasticizers such as but not limited to rayon viscose, starch-based polymer, and acetate; petroleum derivatives and petroleum gels; fats, both synthetic and natural; polyurethane; natural latex; and mixtures and combinations thereof. Each possibility represents a separate embodiment of the invention.

According to some embodiments, the polymer is a synthetic polymer, including organic polymers, inorganic polymers and bioplastics. According to some embodiments, the polymer is selected from the group consisting of polyamide, polyester, acrylic, polyolefin, polysiloxane, nitrile, polyvinyl acetate, cellulose-based polymers, starch-based polymer, derivatives, dispersions and combinations thereof. Each possibility represents a separate embodiment of the invention. The non-limiting examples of the cellulose-based polymer are viscose or rayon. According to certain embodiments, the polymer is selected from the group consisting of polyamide, polyester, acrylic, polyalkene and combinations thereof. According to other embodiments, the polymer is selected from the group consisting of polyamide, polyalkene, polyurethane, polyester and combinations thereof. Each possibility represents a separate embodiment of the invention. According to particular embodiments, the polymer is selected from polyethylene, polypropylene, acrylonitrile butadiene styrene (ABS), polylactic acid (PLA), polyglycolic acid (PGA), poly(lactic-co-glycolic acid) (PLGA), poly(butyl acrylate) (PBA), polybutylene terephthalate (PBT) and combinations thereof. The polymer may be water based or solvent based.

Combinations of more than one of said materials can also be used provided they are compatible or adjusted for compatibility.

### The Polymer Having the Metal Oxide Powders Incorporated Therein

According to some embodiments, the metal oxide powders are incorporated into the polymer by a master batch manufacturing.

As used herein, the term “master batch” unless otherwise indicated, refers to a carrier polymer containing metal oxide particles, formed into pellets or granules, wherein the polymer is compatible with the end product material. The master batch can be added as a chemical additive to a polymeric slurry comprising same or chemically compatible polymer before extrusion, molding, casting or 3D printing. Alternatively, the master batch can comprise a compounded resin containing the final dosage of the polymers and the metal oxides required for the product to be formed from the polymer.

Metal oxide powders can be included in a polymer using a master batch system so that the powder particles form part of the entire polymeric product. However, the currently known processes for the preparation of a polymeric material having skin-regenerating properties are adapted for inclusion of a single type of metal oxide. The present invention provides materials comprising a combination of a mixed oxidation state oxide of a first metal and a single oxidation state oxide of a second metal. According to some exemplary embodiments, the first metal and the second metal are different. Thus, according to further embodiments, the at least two metal oxide powders have substantially different specific gravities.

When two or more particulate compounds having different specific gravities and being disruptive to non-isotactic materials, such as the majority of polymers, have to be incorporated into the polymeric material, control over suspension and dispersion of the particles in the polymeric slurry is complicated. Such slurries generally yield inhomogeneous extruded or cast polymers. Dispersion and suspension of distinct metal oxide powders is not usually practiced in master batch production, where normally a specific single compound is desired to be added to the polymer. Therefore, when reducing the invention to practice, it was required to develop a method allowing incorporation of at least two metal oxide powders having substantially different specific gravities into a polymer fiber. Furthermore, since the amount of any metal oxide powder that can be incorporated into a polymer is limited by the disruption effect of the metal oxide on cross polymerization of non-isotactic polymers or weakening of the carrier polymer, it was necessary to develop a method to accommodate a high amount of multiple metal oxides in these polymers. The present invention thus provides a process for the preparation of the material having skin-regenerating properties, providing control over the metal oxide particles concentration and distribution in the polymer. The present invention further provides materials having skin-regenerating properties, comprising a combination of at least two metal oxide powders, wherein the metal oxide powders are incorporated within the polymer fiber in a generally uniform fashion. As used herein, the terms “generally uniform” or “homogeneous” that can be used interchangeably, denote that the volume percentage of the metal oxide particles on the polymer surface or in the bulk thereof varies by less than about 30%, less than about 20%, less than about 10% or less than about 5%. Each possibility represents a separate embodiment of the invention.

According to some embodiments, the materials of the present invention comprise at least two metal oxide powders having substantially different specific gravities. The term “substantially different specific gravity” refers, in some embodiments, to the variance in the specific gravities of the at least two metal oxide powders, which is higher than about 5%. In another embodiment, the term refers to the variance of higher than about 10%. In yet another embodiment, the term refers to the variance of higher than about 15%.

To accommodate a plurality of metal oxide powders having distinct specific gravities in a single polymeric slurry, it is necessary to compensate for the particle weight differences of the metal oxides. In order to do so, the bulk densities of the metal oxide powders should be equalized. As used herein, the term “bulk density” refers to the mass of many particles of the powder divided by the total volume they occupy. According to some embodiments, the material comprises at least two metal oxides powders processed to have a substantially similar bulk density. “Substantially similar bulk density” refers, in some embodiments, to the variance in the bulk density of the at least two metal oxide powders, which is less than about 20%. In another embodiment, the term refers to the variance of less than about 10%. In yet another embodiment, the term refers to the variance of less than about 5%. In some embodiments, the at least two metal oxide powders have substantially similar bulk densities.

For example, specific gravity of copper oxide is 6.0 g/ml, wherein specific gravity of tetrasilver tetroxide is 7.48 g/ml. The bulk densities of the unprocessed copper oxide and the tetrasilver tetroxide powders are thus significantly different. Without wishing to being bound by theory or mechanism of action, in order to be incorporated into the polymer in a substantially uniform manner, the powders have to be processed to equalize the bulk densities thereof. Equalizing the bulk densities of the metal oxide powders can be achieved by altering the particle size of the metal oxide powders. Said particle size alteration can be performed by decreasing or increasing the particle size of the powders. For example, the particles size of the powders can be decreased by grinding and increased by applying a coating. The extent of the increase or decrease in the particle sizes of one metal oxide powder as compared to the other metal oxide powder is dependent on the specific gravities and/or the initial bulk densities of said metal oxide powders.

According to some embodiments, the metal oxide powders are processed by grinding. According to certain embodiments, the metal oxide powders are processed to have mean particle sizes which are inversely proportional to the specific gravities thereof. According to another embodiment, the metal oxide powders are ground to have mean particle sizes which are inversely proportional to the specific gravities thereof. According to the further embodiments, the mean particle sizes of the metal oxide powders are inversely proportional to the specific gravity thereof.

According to further embodiments, the material comprises at least two metal oxide powders having essentially similar particle sizes. “Substantially similar particle size” refers, in another embodiment, to the variance in the particle size of the at least two metal oxide powders which is less than about 20%. In another embodiment, the term refers to the variance of less than about 10%. In yet another embodiment, the term refers to the variance of less than about 5%. In still another embodiment, the term refers to the variance of less than about 1%.

According to further embodiments, the metal oxide powders are processed to have substantially similar particle sizes. According to further embodiments, the metal oxide powders are ground to have substantially similar particle sizes. According to yet further embodiments, at least one of the metal oxide powders is ground to obtain the at least two metal powders having substantially similar particle sizes.

According to some embodiments, the particles of at least one metal oxide powder comprise a coating. According to other embodiments, the particles of at least two metal oxide powders comprise the coating. In some embodiments, at least one of the metal oxide powders is processed to have coated particles. In further embodiments, each of the at least two metal oxide powders is processed to have coated particles. According to certain embodiments, said particles have substantially similar sizes. According to further embodiments, the coating thickness is proportional to the specific gravity of the metal oxide powders. According to yet further embodiments, the coating weight is proportional to the specific gravity of the metal oxide powders. According to some embodiments, the at least two metal oxide powders comprise particles having a different coating material. The molecular or specific weight of the coating material can be adjusted to compensate for the difference in the specific gravities of the metal oxide powders.

The metal oxide particles coating may comprise polyester or polyalkene wax. The non-limiting examples of the polyalkene wax include polypropylene wax marketed by Clariant as Licowax PP 230, an oxidized polyethylene wax marketed by Clamant as Licowax PED 521, an oxidized polyethylene wax marketed by Clariant as Licowax PED 121 or an ethylene homopolymer wax marketed by BASF as Luwax®.

According to further embodiments, the coating material comprises a copolymer of polyethylene wax and maleic anhydride. According to yet further embodiments, the coating material further comprises ionomers of low molecular weight waxes. According to additional embodiments, the polyethylene wax has a high wettability. In some embodiments, the coating material comprises homopolymers, oxidized homopolymers, high density oxidized homopolymers and co-polymers of polyethylene, polypropylene and ionomer waxes, micronized polyalkene waxes or mixtures thereof, as well as co-polymers of ethylene-acrylic acid and ethylene-vinyl acetate.

A critical prerequisite for the usability of such an additive concentrate is the correct choice of the wax component. Although it is not colored itself, it influences the performance of the additive concentrate. For more detailed information, reference may be made, for example, to the product brochure “Luwaxe®—Anwendung in Pigmentkonzentraten” about polyethylene waxes from BASF AG.

According to some embodiments, the weight of the coating material applied to the powder constitutes from about 0.2% to about 2% wt. of the metal oxide powder weight. According to additional embodiments, the weight of the coating material constitutes from about 0.2% to about 1% wt. of the metal oxide powder weight, preferably from about 0.4 to about 0.5% wt. Each possibility represents a separate embodiment of the invention. In a certain embodiment, the weight of the coating material constitutes about 1% wt. of the metal oxide powder weight.

According to other embodiments, the first metal and the second metal are the same. According to further embodiments, the at least two metal powders have substantially similar bulk densities. Without wishing to being bound by theory or mechanism of action, in order to hinder a chemical interaction between the metal oxide powders and the carrier polymer or the polymer fiber, the metal oxides should be pretreated with an encapsulating compound. Said compounds isolate the metal oxides so that they will not interact with the polymeric material and are configured to abrade off the powder during product use. Thus, according to some embodiments, the materials of the present invention comprise metal oxide powders, comprising particles encapsulated within an encapsulating compound. The encapsulating compound can be selected from the group consisting of silicates, acrylates, cellulose, protein-based compounds, peptide-based compounds, derivatives and combinations thereof. In some embodiments, the encapsulating compound is selected from the group consisting of silicate, poly(methyl methacrylate) (PMMA) and a combination thereof.

According to some embodiments, the weight of the encapsulating compound applied to the powder constitutes from about 0.2% to about 2% wt. of the metal oxide powder weight. According to additional embodiments, the weight of the encapsulating compound constitutes from about 0.2% to about 1% wt. of the metal oxide powder weight, preferably from about 0.4 to about 0.5% wt. Each possibility represents a separate embodiment of the invention.

Additionally or alternatively, the chemical interaction between the metal oxide powders and the carrier polymer or the polymeric support, can be hindered through addition of metal deactivating agents or chelating agents. As used herein, the terms “metal deactivating agents” and “chelating agents” that can be used interchangeably, refer to an agent generally comprising organic molecules containing heteroatoms or functional groups such as a hydroxyl or carboxyl, the agent acting by chelation of the metal to form inactive or stable complexes.

Thus, according to some embodiments, the materials of the present invention comprise a metal deactivating agent or a chelating agent. In further embodiments, the materials of the present invention comprise a metal deactivating agent or a chelating agent associated with the metal oxide powders. The non-limiting example of the said metal deactivating agents and/or chelating agents include a phenolic antioxidant, potassium iodide, potassium bromide, calcium stearate, zinc stearate, aluminum stearate, tertiary chain extenders and combinations thereof. According to a particular embodiment, the metal deactivating agent is a phenolic antioxidant. The phenolic antioxidant can be selected from, but not limited to 2′,3-bis [[3-[3,5-di-tert-butyl-4-hydroxyphenyl] propionyl]] propionohydrazide marketed under the name Irganox® MD 1024 by CIBA; Vitamin E (alpha-tocopherol) which is a high molecular weight phenolic antioxidant, marketed under the name Irganox® E 201 by CIBA; Irganox® B 1171, marketed by CIBA, which is a blend of a hindered phenolic antioxidant and a phosphate; and combination thereof. According to certain embodiments, the metal deactivating agents abrade off the metal oxide particles upon hydration of the material.

According to some embodiments, the weight of the metal deactivating agent applied to the powders constitutes from about 0.2% to about 5% wt. of the metal oxide powder weight. According to additional embodiments, the weight of the metal deactivating agent comprises from about 0.5% to about 1% wt. of the metal oxide powder weight. In a certain embodiment, the weight of the metal deactivating agent constitutes about 1% wt. of the metal oxide powder weight.

Another difficulty in adding almost any inorganic compound to a polymeric material is particle agglomeration. According to some embodiments, the metal oxide particles of the present invention are treated by a surfactant to prevent metal oxide particles agglomeration. Therefore, according to some embodiments, the materials of the present invention comprise a surfactant. In further embodiments, the materials comprise a surfactant associated with the metal oxide powders. The non-limiting examples of the surfactant include but are not limited to Sigma Aldrich Niaproof®, Dow Corning Xiameter® and Triton-X-100.

According to some embodiments, the weight of the surfactant constitutes from about 0.05% to about 2% wt. of the metal oxide powder weight. In a certain embodiment, the weight of the surfactant constitutes about 0.5% wt. of the metal oxide powder weight.

According to some embodiments, the metal oxide powders are present in the master batch at a weight percent of from about 0.5% to about 95% of the total weight of the master batch. According to further embodiments, the metal oxide powders are present in the master batch at a weight percent of from about 5% to about 50%, preferably from about 20% to about 40%. Each possibility represents a separate embodiment of the invention. According to some embodiments, the master batch is prepared for direct extrusion, molding or casting, without further mixing with an additional polymer. In certain such embodiments, the metal oxide powders are present in the master batch at a weight percent of from about 0.5% to about 30% of the total weight of the master batch, preferably from about 0.5% to about 15% of the total weight of the master batch. According to further embodiments, the metal oxide powders are present in the master batch in an amount configured to provide from about 0.5% wt. to about 30% wt. of the metal oxide particles in the material obtained through a master batch manufacture process, preferably from about 0.5% wt. to about 15% wt., or from about 1% wt. to about 5% wt. of the metal oxides of the total weight of the material. Each possibility represents a separate embodiment of the invention.

The composition of the master batch, comprising the polymer and the synergistic composition of the metal oxide powders can be formed into a semi-final or a final product. The semi-final product may include, inter alia, a fiber, a yarn, a textile, a fabric, a film or a foil; and the final product may include, inter alia a textile product or a non-textile polymeric article. According to some embodiments, the fiber is a polymeric fiber, being synthetic or semi-synthetic. The fiber may be a staple fiber or a filament fiber. According to some embodiments, the master batch composition is formed into a semi-final or final product by means of extrusion, molding, casting or 3D printing of the polymer, comprising said synergistic combination. According to further embodiments, the material is selected from an extruded, molded, cast or 3D printed polymer. Each possibility represents a separate embodiment of the invention.

Thus, in some embodiments, the metal oxide powders are present in the master batch in an amount configured to provide from about 0.5% to about 30% wt. of the metal oxide particles in the extruded or molded polymer obtained through a master batch manufacture process, preferably from about 0.5% wt. to about 15% wt., or from about 1% wt. to about 5% wt. of the metal oxides of the total weight of the extruded or molded polymer. Each possibility represents a separate embodiment of the invention. In still further embodiments, the metal oxide powders are present in the master batch in an amount configured to provide from about 0.5% to about 30% wt. of the metal oxide particles in the polymer fiber obtained through a master batch manufacture process, preferably from about 0.5% wt. to about 15% wt., or from about 1% wt. to about 5% wt. of the metal oxides of the total weight of the total weight of the polymer fiber. Each possibility represents a separate embodiment of the invention.

In some embodiments, the combined weight of the at least two metal oxides constitutes from about 0.25% to about 50% wt. of the total weight of the materials.

In some embodiments, the material is in a form of a semi-final product, selected from a fiber, a yarn, a textile, a fabric, a film or a foil. In certain such embodiments, the combined weight of the at least two metal oxides constitutes from about 0.5% to about 30% wt. of the total weight of the semi-final product.

According to further embodiments, the combined weight of the at least two metal oxides constitutes from about 1% to about 15% wt. of the total weight of the semi-final product. In certain such embodiments, the polymer is selected from polyester or polyamide. In further embodiments, the semi-final product is a fiber, preferably including a staple fiber. According to certain embodiments, the combined weight of the at least two metal oxides constitutes from about 1% to about 5% wt. of the total weight of the semi-final product. According to other embodiments, the combined weight of the at least two metal oxides constitutes from about 3% to about 8% wt. of the total weight of the semi-final product.

According to further embodiments, the combined weight of the at least two metal oxides constitutes from about 0.5% to about 8% wt. of the total weight of the semi-final product. In certain such embodiments, the polymer is selected from polyester or polyamide. In further embodiments, the semi-final product is a fiber, preferably including a filament fiber. According to certain embodiments, the combined weight of the at least two metal oxides constitutes from about 1% to about 4% wt. of the total weight of the semi-final product. According to other embodiments, the combined weight of the at least two metal oxides constitutes from about 0.5% to about 2% wt. of the total weight of the semi-final product.

According to further embodiments, the combined weight of the at least two metal oxides constitutes from about 10% to about 30% wt. of the total weight of the semi-final product. In certain such embodiments, the polymer is a polyalkene. In further embodiments, the semi-final product is a fiber.

In some embodiments, the combined weight of the at least two metal oxide powders constitutes from about 3% to about 8% wt. of the total weight of material and the size of the metal oxide particles is from about 0.5 to about 1 micron. In particular embodiments, the combined weight of the at least two metal oxide powders constitutes about 3% wt. of the total weight of material, wherein the metal oxide particles size is about 1 micron. In other embodiments, the combined weight of the metal oxides powders constitutes about 8% wt. of the total weight of material, wherein the metal oxide particles size is about 0.5 micron.

In some embodiments, the polymer fiber is blended with a natural fiber. The natural fiber may be selected from the group consisting of cotton, silk, wool, linen and combinations thereof. In additional embodiments, the material further comprises a modified cellulose fiber. The non-limiting examples of cellulose modified fiber include viscose and rayon.

According to some embodiments, the natural fiber may be present in the material in a weight percent of up to about 95% of the total weight of the material. In further embodiments, the material of the present invention comprises from about 50% to about 85% wt. of natural fiber. According to some exemplary embodiment, the natural fiber may be present in the material in a weight percent of about 70% of the total weight of the material. According to further embodiments, the weight ratio between the polymer fiber with at least two metal powders incorporated therein and the natural fiber is from about 1:1 to about 1:6. The blend material therefore may comprise from about 50% wt. natural fiber/50% wt. polymer fiber to about 85% wt. natural fiber/15% wt. polymer fiber. In certain embodiments, the material comprises from about 50% wt. cotton/50% wt. polymer fiber to about 85% wt. cotton/15% wt. polymer fiber.

According to some embodiments, the material comprises a semi-final product comprising to blend of a polymeric fiber and a natural fiber. In certain such embodiments, the combined weight of the at least two metal oxides constitutes from about 0.25% to about 5% wt. of the total weight of the semi-final product.

In some embodiments, the material comprises 100% wt. polymer fiber with at least two metal powders incorporated therein. Therefore, the material of the present invention can be devoid of natural fibers.

According to some embodiments, the weight of the mixed oxidation state oxide constitutes from about 0.125% to about 25% wt. of the total weight of the material. In some embodiments, the mixed oxidation state metal oxide constitutes from about 0.5% to about 15% wt. of the total weight of the material, preferably from about 1% to about 5% wt. of the total weight of the material. Each possibility represents a separate embodiment of the invention. The material can be selected from an intermediate, semi-final and final material.

According to some embodiments, the material having cell proliferation properties comprises the polymer and a synergistic combination of the at least two metal oxide powders, wherein the powders are incorporated within the polymer. According to some embodiments, the metal oxides powders are attached to the polymer. According to further embodiments, the powders are attached to the polymer surface. According to other embodiments, the powders are embedded into the polymer. According to further embodiments, the powders are embedded into the polymer surface. According to other embodiments, the powders are deposited on the polymer surface. According to additional embodiments, the powders are inserted into the polymer. According to further embodiments, the powders are inserted into the polymer surface. According to further embodiments, the metal oxide powders particles protrude from the polymer surface. According to some embodiments, at least 10% of the synergistic combination of the metal oxides is present on the surface of the polymer. According to further embodiments, at least 5% of the synergistic combination of the metal oxides is present on the surface of the polymer. It has been found that as little as 1% appearance on the surface of a polymeric fiber, which contains particles protruding from the polymer surface, was sufficient to ensure a biocidal effect. Thus, according to some embodiments, at least 1% of the synergistic combination of the metal oxides is present on the surface of the polymer. According to other embodiments, the powders are not exposed on the surface of the polymer. According to some embodiments, said polymer is an extruded, cast or molded polymer or is in a form of a polymer fiber, a textile product or a non-textile polymeric article. Each possibility represents a separate embodiment of the invention.

### The End Product

The materials of the present invention comprise a polymer and at least two metal powders incorporated therein, wherein the polymer can be an extruded, molded, cast or 3D printed polymer. According to some embodiments, said polymer is a molded polymer. In other embodiments, the polymer is an extruded polymer. In certain embodiments, the polymer is a form of a fiber. According to some embodiments, the fiber can be formed into a yarn, textile or fabric. According to some embodiments, the textile is selected from a woven textile, a knit textile, a non-woven textile, a needle-punch textile or felt.

Also provided according to some embodiments of the present invention is an extruded polymer in the form of a staple or a filament fiber, comprising said metal oxide particles incorporated into a polymer fiber and formed as a non-extruded non-woven material or filling.

According to further embodiments, the semi-final product can be formed into a final product. According to some embodiments, final products of the present invention have a soft surface. The term “soft surface” as used herein, refers to all surfaces which are solid but are not hard surfaces, and most often refers to products made from knit, woven, or non-woven textile products. The final products of the present invention include, but are not limited to, textile products and non-textile polymeric articles.

The textile product may be selected from, but not limited to, clothing items, bedding textiles, medical textiles including bandages or sutures and textiles for internal and external use. The non-limiting examples of the textile products include pillowcases, eye-masks, gloves, socks, stockings, sleeves, undergarments, sheets, bedding, gauze pads, trans-dermal patches, bandages, adhesive bandages, sutures, compression garments in all sizes for different parts of the body, sheaths and textiles for internal use.

The non-textile polymeric article may be selected, but not limited to, medical textiles including bandages or sutures, mono-lithic films, breathable films, compression garments in all sizes for different parts of the body, and absorbent pads.

Products formed from the materials of the present invention, possess effective cell proliferation properties, including, but not limited to skin-regeneration and would healing.

Thus, according to some embodiments, the materials of the present invention are for use in enhancing mammalian cell proliferation. In further embodiments, the material is for use in skin regeneration processes, selected from the group consisting of wound healing, accelerated wound closure, and wound healing with reduced scarring. Each possibility represents a separate embodiment of the invention. The wound can be a naturally caused wound or a surgically-induced wound. In further embodiments, the wound is cutaneous or subcutaneous.

According to some embodiments, the present invention provides a method for inducing a skin regeneration process, the method comprising applying to the skin of a subject in need of such skin regeneration treatment the material according to the principles of the invention. The skin regeneration can include wound healing, accelerated wound closure, or wound healing with reduced scarring.

According to yet further embodiments, the material is for use in cosmetic applications, selected from the group consisting of reduction of wrinkles, reduction of small skin defects, reduction of erythema, reduction of edema, softening of skin and reduction of odor.

According to additional or alternative embodiments, the present invention provides a method for improving skin appearance and/or feel, the method comprising applying to the skin of a subject the material according to the principles of the invention. Improvement of the skin appearance and/or feel can include reduction of wrinkles, reduction of small skin defects, reduction of erythema, reduction of edema, softening of skin or reduction of odor.

### Preparation Method

In another aspect, the present invention provides a method for the preparation of the material according to the principles of the present invention, the method comprising mixing the at least two metal oxide powders with at least one polymer.

In some embodiments, the mixed oxidation state oxide constitutes from about 25% wt. to about 75% wt. of the total weight of the synergistic combination of the at least two metal oxide powders

In some embodiments, the at least two metal oxide powders have substantially different specific gravities, In further embodiments, the method comprises processing the at least two metal oxide powders to have substantially similar bulk densities prior to mixing thereof with the polymer. According to some embodiments, the method comprises processing the metal oxide powders to obtain particles having sizes which are inversely proportional to the specific gravity thereof. According to some embodiments, the method comprises reducing the metal oxide powders particle size to obtain particles having sizes which are inversely proportional to the specific gravity thereof. According to other embodiments, the method comprises processing the metal oxide powders to obtain particles having substantially similar sizes. According to other embodiments, the method comprises reducing the metal oxide powders particle size to obtain particles having substantially similar sizes. In some embodiments, said processing comprises grinding.

In additional embodiments, the method further comprises applying a coating to the metal oxide powder particles. According to some embodiments, the coating thickness is proportional to the specific gravity of the metal oxide particles. According to other embodiments, the coating weight is proportional to the specific gravity of the metal oxide particles. According to some embodiments, the coating is applied to metal oxide powders having substantially similar particle sizes. According to further embodiments, the coating is applied to at least one metal oxide powder. According to other embodiments, the coating is applied to at least two metal oxide powders. According to further embodiments, the coating comprises polyester or polyalkene wax. The polyester or polyalkene wax may be selected from the group consisting of a polypropylene wax, oxidized polyethylene wax, ethylene homopolymer wax, and different types of waxes including copolymers of polyethylene wax and maleic anhydride which can also be used with ionomers of low molecular weight waxes or any combination thereof.

In some embodiments, the method further comprises a step of encapsulating the metal oxide powder particles within an encapsulating compound. In other embodiments, the method comprises a step of mixing the metal oxide powders with a metal deactivating agent or a chelating agent. In further embodiments, the method comprises a step of mixing the metal oxide powders with a surfactant. In some embodiments, the additional steps are performed prior to mixing the metal oxide powders with the polymer.

The encapsulating compound may be selected from the group consisting of silicate, acrylate, cellulose, derivatives thereof and combinations thereof. The metal deactivating agent may be selected from the group consisting of phenolic antioxidant, potassium iodide, potassium bromide, calcium stearate, zinc stearate, aluminum stearate, tertiary chain extender and a combination thereof.

In additional embodiments, the method comprises preparing the mixed oxidation state oxide. The mixed oxidation state oxide can be prepared by a standard procedure, for example as described by Hammer and Kleinberg in Inorganic Synthesis (IV,12) or in U.S. Pat. No. 5,336,416, which are incorporated by reference herein in their entirety. The method may further include a step of grinding the obtained mixed oxidation state oxide powder.

According to some embodiments, the mixing of the metal oxide powders and the at least one polymer is assisted by sonication.

According to further embodiments, the mixing of the at least two metal oxide powders with the polymer comprises producing a master batch, comprising the metal oxide powders and a carrier polymer. According to some embodiments, said at least one polymer comprises the carrier polymer. According to the preferred embodiments, the master batch is homogeneous. According to additional embodiments, the metal oxide powders are distributed in the master batch in a generally uniform manner. The master batch may be formed into pellets. Alternatively, the master batch may be formed into granules. The carrier polymer may be selected from the group consisting of polyamide, polyalkene, polyurethane, polyester and combinations thereof.

In some embodiments the mixing of the at least two metal oxide powders with the polymer further comprises adding the master batch to a polymer slurry. In further embodiments the polymer slurry comprises a polymer, which is the same as the carrier polymer. In other embodiments, the polymer slurry comprises a polymer, which is chemically compatible with the carrier polymer. In some embodiments, the polymer is selected from the group consisting of polyamide, polyalkene, polyurethane and polyester. Combinations of more than one of said materials can also be used provided they are compatible or adjusted for compatibility. The polymeric raw materials are usually in bead form and can be mono-component, bi-component or multi-component in nature. The beads are heated to melting at a temperature which preferably will range from about 120 to 180° C. for isotactic polymers and up to 270° C. for polyester. The master batch is then added to the polymer slurry and allowed to spread through the heated slurry. The particle size of the metal oxide powders in these embodiments is preferably between 1 and 5 microns. However particulate size can be larger when the film or fiber thickness can accommodate larger particles. According to other embodiments, particle size of the metal oxide powders is between 0.1 and 0.5 microns. According to further embodiments, the metal oxide powders are sonicated prior to incorporation into the polymer fiber.

According to some embodiments, the method further comprises forming from the obtained mixture a semi-final or a final product. Thus, in some embodiments, the method further comprises comprising forming from the obtained mixture a film, a foil, a fiber, a yarn, a fiber or a textile, comprising said powders. Each possibility represents a separate embodiment of the invention. According to further embodiments, the method comprises a step of forming the film, foil, fiber, yarn, fiber or textile into a textile product or a non-textile polymeric article,

In some embodiments, the step of forming a final or semi-final product comprises extrusion, molding, casting or 3D printing of the mixture of the metal oxide powders with the polymer. In some exemplary embodiments said step comprises extrusion. In certain such embodiments, the polymer slurry is transferred to an extrusion tank. In further embodiments, the liquid polymer slurry is pushed through holes in a series of metal plates formed into a circle called a spinneret. The polymer slurry is pushed through a spinneret by applying pressure on the slurry. As the slurry is pushed through the fine holes that are close together, they form single fibers or if allowed to contact one another, they form a film or sheath. The hot liquid fiber or film is pushed upwards, cooled with cold air, forming a continuous series of fibers or a circular sheet. The thickness of the fibers or sheet is controlled by the size of the holes and speed at which the slurry is pushed through the holes and upward by the cooling air flow. In the preferred embodiments, the fibers are homogeneously extruded.

In some embodiments, the step of forming a final or semi-final product comprises forming a polymeric fiber from the mixture of the metal oxide powders with the polymer. The formation of a fiber can be in either filament form (continuous) or staple form (short cut). In both cases an amount of master batch is added to the hot polymeric slurry to yield the final amount of the combination of the at least two metal oxide powders desired for the end product. By way of example if a 1% final load is desired in a filament fiber than 50 kilo of a 20% wt. concentrated master batch will be added to complete 1 ton of total slurry. By way of example if a 3% final load is desired in a staple fiber than 150 kilo of a 20% wt. concentrated master batch will be added to complete 1 ton of total slurry. In both cases, after a thorough mixing of the concentrated master batch in the slurry tub to obtain good master batch dispersion, the extruded fibers will contain the desired amount of the metal oxides combination.

In a normal process as known to those familiar with the art, the active ingredient will be evenly dispersed and remain in suspension of the polymeric slurry. If the master batch is not prepared correctly then the metal oxides will interact with the target polymer and disrupt the linkage process thus inhibiting the formation of a solid fiber. In addition, if the wax is not applied correctly the metal oxides will either sink to the bottom of the mixing tub and block the holes of the spinneret or will remain floating at the top of the slurry and not get mixed into the fibers. Normally extrusion is done using gravity so that the weight of the slurry in the tub pushes the polymer through the spinneret holes. The polymer is designed to solidify with exposure to air. Once the fibers are exposed to air they are wound on bobbins for further processing.

According to some embodiments, the fiber is selected from the group consisting of a staple fiber, a filament fiber and a combination thereof. According to some embodiments, the polymer fiber is a synthetic or a semi-synthetic fiber. According to further embodiments, the synthetic or semi-synthetic fiber is selected from the group consisting of polyolefin fibers, polyurethane fibers, vinyl fibers, nylon fibers, polyester fibers, acrylic fibers, cellulose fibers, regenerated protein fibers, blends and combinations thereof. In some embodiments, the method further comprises blending the polymer fiber with a natural fiber. According to further embodiments, the natural fibers are selected from the group consisting of cotton, silk, wool, linen and combinations thereof.

According to further embodiments, the method includes forming the polymer fiber into a yarn. According to some embodiments, the yarn is a synthetic yarn or a combination of the synthetic yarn with a natural yarn. In some embodiments, the synthetic yarn is spun from said synthetic fibers. According to further embodiments, the yarn is formed into fabrics. According to further embodiments, the fabrics are woven, knit or non-woven.

In additional embodiments the method further comprises forming the material into a textile product or a non-textile polymeric article. According to further embodiments, step c., comprises directly forming from the mixture obtained in step b. a textile product or a non-textile polymeric article. Each possibility represents a separate embodiment of the invention. In certain such embodiments, step c includes molding, casting or extruding the mixture obtained in step b. into a desired shape or form.

In certain embodiments, step c. comprises applying the mixture of the metal oxide powders with the at least one polymer, obtained in step b. to a pre-formed polymer article as a second layer. In some embodiments, the polymer is latex, nitrile or an artificial rubber.

The following examples are presented for illustrative purposes only and are to be construed as non-limitative to the scope of the invention.

### EXAMPLES

### Example 1: Mixed Oxidation State Oxide Powder Preparation

A tetrasilver tetroxide powder was prepared through a reduction process from a silver nitrate solution by a standard procedure known to a person skilled in the art, and as described by Hammer and Kleinberg in Inorganic Synthesis (volume IV, page 12). It should be further noted that the powder obtained by the described process should be very soft and capable of being converted into a nano-powder with a relative ease.

The basic tetrasilver tetroxide (Ag₄O₄) synthesis as referenced above was prepared by addition of NaOH into distilled water, followed by addition of a potassium persulfate and then the addition of silver nitrate.

A tetracopper tetroxide powder can be prepared using copper sulfate and potassium persulfate as an oxidizing agent, as described in U.S. Pat. No. 5,336,416 to Antelman. However, for the sake of commercial viability cuprous oxide was purchased and used as a starting material to obtain Cu₄O₄ according to the described procedure.

The particle size of both powders received varies from nano-particles to agglomerated particles as large as 20 microns.

These powders can be ground down to the desired particle size and mixed either together or with copper oxide or zinc oxide. The copper oxide used in the development is a cuprous oxide (brown/red) with a purity level of no less than 97% in a 10-20 μm size particle. In this case, the powder was purchased from SCM Inc. of North Carolina, USA, but can be purchased from any supplier who can furnish this purity level. The powder is then ground down to 1 to 5 μm. Due to the prevalence of suppliers of this powder it is not economically viable to manufacture the powder. However, due to the difficulty in obtaining tetrasilver tetroxide and/or tetracopper tetroxide, it is necessary to synthesize the specific species as described hereinabove.

### Example 2: Master Batch Preparation

The metal oxides were incorporated into a polymer using a master batch system so that the powder is embedded on the outside of the polymer and forms part of the entire polymeric product.

To accommodate the different specific gravities of more than one metal oxide in a common master batch, it is necessary to compensate for the differences between the two different metals, should a difference in their weight exist. This is done using two systems as described:

In the first system, the particle sizes of each metal oxide were made equal through proportional size equalization. The specific gravity of copper oxide is approximately 6 g/ml and the specific gravity of tetrasilver tetroxide is 7.48 g/ml. Tetrasilver tetroxide particles were ground down to be approximately 10% to 15% smaller than the copper oxide particles.

In the second system, the particles were all ground to the same size but the heavier particles were coated with a higher amount of polyester wax or polyethylene wax.

The wax was applied in a high sheer mixer in a weight/weight ratio of approximately 10 grams wax to 1000 grams metal oxide. It was found that a higher amount of polyester wax on the heavier metal oxide aids in maintaining the suspension of the metal oxide in the polymer slurry. The wetting capability of the waxes should also be good. To isolate the metal oxide from a chemical interaction with the carrier polymer, the metal oxide powders were pretreated with an encapsulating compound. The inert encapsulating compounds used were a silicate and poly(methyl methacrylate) (PMMA). The encapsulation was performed in a high sheer mixer in a weight/weight ratio of approximately 4 g encapsulating agent to 1000 g metal oxide powder.

### Example 3: Polymer and Blended Polymer Fiber and Yarn Preparation

The fabrication of a polymeric yarn having cell proliferation properties, characterized by a protrusion of the metal oxide particles on the surface of the polymer in both a filament and staple product is described.

It should be noted that for the sake of this example, tetrasilver tetroxide and/or tetracopper tetroxide and/or copper oxide were used but that the proportions for using other metal oxides compounds are the same approximate proportions.

A description of the general production process of fibers is as follows:

1. Slurry is prepared from any polymer, the chief raw material preferably being selected from a polyamide, a polyalkene, polyurethane and polyester. Combinations of more than one of said materials can also be used provided they are compatible or adjusted for compatibility. The polymeric raw materials are usually in bead form and can be mono-component, bi-component or multi-component in nature. The beads are heated to melting at a temperature which preferably will range from about 120 to 180° C. for isotactic polymers and up to 270° C. for polyester.

2. At the hot mixing stage, before extrusion, a water insoluble powder of the chosen metal oxide compounds in the form of master batch is added to the slurry and allowed to spread through the heated slurry. The particulate size will be preferably between 1 and 5 microns, however particulate size can be larger when the film or fiber thickness can accommodate larger particles.

3. The liquid slurry is then pushed with pressure through holes in a series of metal plates formed into a circle called a spinneret. As the slurry is pushed through the fine holes that are close together, they form single fibers or if allowed to contact one another, they form a film or sheath. The hot liquid fiber or film is pushed upwards, cooled with cold air, forming a continuous series of fibers or a circular sheet. The thickness of the fibers or sheet is controlled by the size of the holes and speed at which the slurry is pushed through the holes and upward by the cooling air flow.

### Filament Fiber

It is noted that the specific gravity of each metal oxide is different and therefore required a treatment of a different coating compound or applying different amount of the same coating compound so that both metal oxide powders would be homogeneously dispersed in the liquid polyester slurry. The metal oxide particles were mixed with the carrier and formed into pellets. As it relates to filament fiber this produces a total of 50 kilo of master batch which is a total of the copper oxide and/or the tetrasilver and/or tetracopper tetroxide is together. The proportion of the carrier to active material was 5:1 yielding a 20% wt. concentration of the metal oxides in the master batch. 50 kilo of the master batch were mixed into an extrusion tank for spinning through a spinneret and were sufficient to produce 1 ton of a filament polymeric yarn yielding a total of a 1% final concentration of the two metal oxides (active material) together in the polymer yarn. It should be noted that if the particles are below 0.5 microns in size it was found that the loading of the metal oxides in a filament fiber can be increased to as much as 4% wt.

### Staple Fiber

For the production of a staple polymeric fiber having mammalian cell proliferation properties, 15 kilo of copper oxide ground to a particle size of 1 to 5 microns and 15 kilo of tetrasilver tetroxide also ground to 1 to 5 microns were mixed with 120 kilo of the chosen carrier polyester polymer for the creation of a master batch. The specific gravity of each compound was different and therefore required a coating by a different coating compound, such as Clamant Licowax PP230 and BASF Luwax® or by different amounts of said compounds, such that the metal oxide particles would be homogeneously dispersed in the suspension. The compounds were mixed with the carrier and were formed into pellets. This produced a total of 150 kilo of master batch. The 150 kilo of master batch was mixed into an extrusion tank for spinning through a spinneret and was sufficient to produce 1 ton of a polymeric staple yarn yielding a total of a 3% wt. final concentration of the two metal oxides in the polymer fiber which appears to be the natural limit for particles above 1 micron in size. It was found that no more than this amount could be easily introduced into a staple fiber without further synthesis. In a staple fiber the normal limit is around 3% wt. but it was found that when the metal oxide particles were reduced to 0.5 micron it was possible to load the fibers with as much as 8% wt.

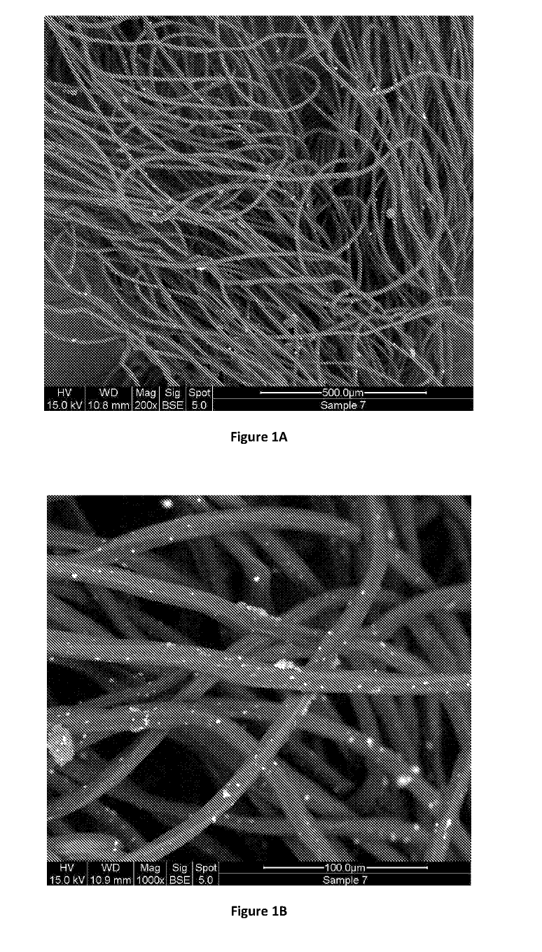

FIGS. 1A-1B present Scanning Electron Microscope (SEM) micrographs of a polyester staple fiber having a combination of copper oxide and tetrasilver tetroxide powders incorporated within. The polymer fiber was prepared by a master batch process as described hereinabove. It can be seen that the metal oxide particles are uniformly distributed on the surface of the polymer fiber. It can also be seen that the metal oxide particles of the synergistic combination protrude from the surface of said polymer fiber.

It has been further found that the load level in a polyolefin fiber can be much higher than in a polyester or nylon fiber due to the isotactic nature of the olefins. While load levels as discussed above were limited to 1% wt. in filament fibers and 3% wt. in staple fibers, it was found that as much as 20% wt. could be added to polypropylene fibers.

Staple and Filament Fiber Combined with Cotton.

A master batch was created as described hereinabove, using polyester resins to which 20 wt. of a mixture comprising copper oxide and TST was added. Both the copper oxide and the TST were about 98% pure. The metal oxide composition comprised 50% copper oxide and 50% wt. TST. The master batch was then added to a polyester slurry being in a liquid form in a proportion that yielded a final loading of copper oxide of about 3% wt. in the final fiber. The copper oxide in the sample was 97.7% pure with 2.3% being impurities. The fibers were extruded in the same manner as normal staple polyester fibers and were then blended with cotton so that the final load of treated fibers is a total of 30% wt. copper oxide and TST impregnated fibers/70% cotton in a 24/1 s forming a ring spun combed cotton yarn twisted for knitting. The yarns were then knit into a fabric that weighs 150 grams to the square meter.

### Example 4: Polymer Yarn Preparation Through a Standard Extrusion Process

The same procedure as in Example 3 is followed for the preparation of the master batch but the yarn is obtained through a procedure involving standard film extrusion equipment. The viscosity of the slurry is controlled through the master batch flow rates, by a procedure which is known to those familiar with the art.

### Example 5: Molded Polymer Preparation

The powders and the master batch including the combined powders including copper oxide and TST, and polypropylene were prepared as described hereinabove. The master batch has to accommodate the carrier polymeric slurry into which it will be added. In molded and/or cast products the master batch concentration can be any amount up to and including 40% active ingredient however, amounts of 20% to 25% would be preferred so as to avoid possible problems in the chemical dispersion in the slurry. The master batch was allowed to melt in the polymeric slurry until the slurry is homogenous. No temperature changes were made. The polymeric slurry is then cast into a desired form or extruded to produce a product of a particular shape. The polypropylene slurry was extruded into a polypropylene film having copper oxide and TST incorporated therein.

### Example 6: Cellulose-Based Polymer Yarn Preparation

A rayon slurry or any cellulose slurry (waste of cotton and corn are very popular as a source of cellulose) is mixed with a plasticizer as is known in the industry of the production of these types of fibers. Normally the process involves a number of chemical steps that involve the breaking down of cellulose to very fine mulch of individual cells, adding a plasticizer, and then exposing the slurry to a solidifying process.

A powder made up of a combination of the two metal oxides, including copper oxide and tetrasilver tetroxide, was prepared. The metal powders were thoroughly mixed together and ground down to a particulate size of preferably under 5 μm.

The powder was then added to the cellulose based slurry in a ratio of up to 3% wt. of the powder to the total weight of the slurry. The powder was added exactly at the same time the slurry is being passed through the holes of the spinneret so that the exposure to the acid in the final step of the process is limited to a few seconds as is common in the way these fibers are made.

The resulting slurry was solidified such that the metal oxide particles are homogeneously impregnated throughout the fiber.

### Example 7: Cell Proliferation Properties of the Metal Oxides

The purpose of this study was to evaluate the potential of the test items at various concentrations to promote wound healing and cell proliferation using the in vitro scratch assay in human foreskin fibroblasts. The assay was conducted in two stages: a preliminary assay to screen for the optimal concentrations levels and proliferation assay to evaluate the proliferation effects of the test item.

Human Foreskin Fibroblasts (HFF) tests were performed using BJ, ATCC, Cat. No. CRL-2252 Human Foreskin Fibroblasts.

### Culture Growth Conditions:

Cultures were propagated at 37° C., humidified 5% CO/air in plastic flasks.

HFF growth medium contained: Dulbecco's Modified Eagle's Medium (DMEM), supplemented with 10% wt. FBS (fetal bovine serum), sodium pyruvate, 2 mM L-glutamine, 100 ug/ml streptomycin 100 U/ml penicillin and 1% wt. non-essential amino acids.

HFF treatment medium contained: DMEM, supplemented with 5% wt. FBS, 1 mM sodium pyruvate, 2 mM L-glutamine, 100 ug/ml streptomycin 100 U/ml penicillin and 1% wt. non-essential amino acids.

### Test Materials:

Test Item 1: Tetrasilver tetroxide (TST) 99.9%; and Test Item 2: Copper oxide 99.9%.

### Test Item Preparation:

Test items (tetrasilver tetroxide and copper oxide) were added to HFF treatment growth medium (containing 5% wt. FBS), each to its saturation point. For the treatments, the saturated solutions were diluted 1:10, 1:100, and 1:500 in the growth medium in a supernatant.

### Test Procedure:

HFF cells were thawed and passaged at least once prior to assay performance. Exponentially growing cultures were collected, centrifuged, counted and allowed to settle in 4×5-well plates) and 3×6-well plates (Stage II) at the density that reaches 100% confluence until the next day.

The following day, before the growth medium was gently and slowly removed, the monolayer was scratched to generate a straight line using a sterile 1 ml pipette tip across the center of the well. Another straight line was scratched perpendicular to the first line to create a visible cross shape in each well.

After scratching, the wells were washed with HFF treatment growth medium to remove the detached cells and replaced with 1.5 ml of HFF treatment growth medium containing the test item at 5 different concentrations including a negative control.

The various concentration intervals of each compound were 1:10, 1:100, 1:500 and 1:1000 dilutions.

HFF cells were thawed and passaged at least once prior to assay performance. Exponentially growing culture was collected, centrifuged, counted and seeded in 96-well tissue culture plate at a density of 5000 cells/well.

The plate was incubated overnight at 37° C., humidified 5% CO₂/air., to enable cells adherence to the wells.

The next day, the growth medium was replaced with the test item solutions in 5% and 1% FBS and vehicle control solution to achieve the final concentrations as designated in the following plate plan (final volume of 200 μl)

Plates were incubated for 24 hours at 37° C., humidified 5% CO₂/air.

Following 24 hours, the medium was replaced with fresh growth medium containing 10% of Alamar Blue. The fluorescent signal (excitation 544 nm/emission 590 nm) was measured following 4 hours incubation time.

### Data Evaluation

The time for complete closure of the scratch was recorded and compared to untreated cells.

$$Viability ( \% ) is expressed as \frac{average treated cells \times 100}{average untreated cells ( vehicle )}$$

### Results

### Stage 1: Preliminary Assay:

24 hours following the plate scratching, the following was reported:
Almost complete closures of scratches were observed in the vehicle control group (growth medium). The scratches were hardly seen under the microscope.
Test Item tetrasilver tetroxide diluted 1:10 was cytotoxic to cells since many dead cells were observed. Scratches could be seen with a naked eye.
Test items tetrasilver tetroxide and copper oxide diluted 1:100 and 1:1000 respectively, showed many proliferation centers along the scratch as compared to the control group. The edges of scratches were covered with cells and could hardly be seen. Nevertheless, in all test item treatments the center of scratches was still clearly seen and had fewer cells than the control group.

### Stage II: Proliferation Assay

The percent viability of the test items on HFF cell line following 24 hours of incubation and 4 hours incubation with the Alamar Blue dye:

The viability results are presented in table 1.

TABLE 1
Viability test results.

| Test # | Description | Number of cells (normalized) |
|---|---|---|
| 1 | Control- | 100 |
| 2 | 100% copper oxide | 107 |
| 3 | 100% TST | 85 |
| 4 | 75% copper oxide/25% TST | 113 |
| 5 | 50% copper oxide/50% TST | 120 |
| 6 | 25% copper oxide/75% TST | 112 |

It was surprisingly observed that the TST alone was cytotoxic. The copper oxide alone demonstrated proliferation. However, the TST in combination with the copper oxide showed a significantly greater cell proliferation.

It should be emphasized that as the concentration of the tetrasilver tetroxide was raised and became closer to the copper oxide concentration, the acceleration of cell proliferation increased. Significantly lower concentrations of one component compared to the other demonstrated synergy, but at a lower level.

The use of copper oxide in wound healing is known, as is the use of tetrasilver tetroxide. But the combination of the two has now been demonstrated to be a cause of accelerated efficacy of cell proliferation, which is related to wound healing and should therefore be an effective combination for wound healing.

### Example 8: Healing Stages of Wounds Treated by Polymer Fiber Including Copper Oxide and TST

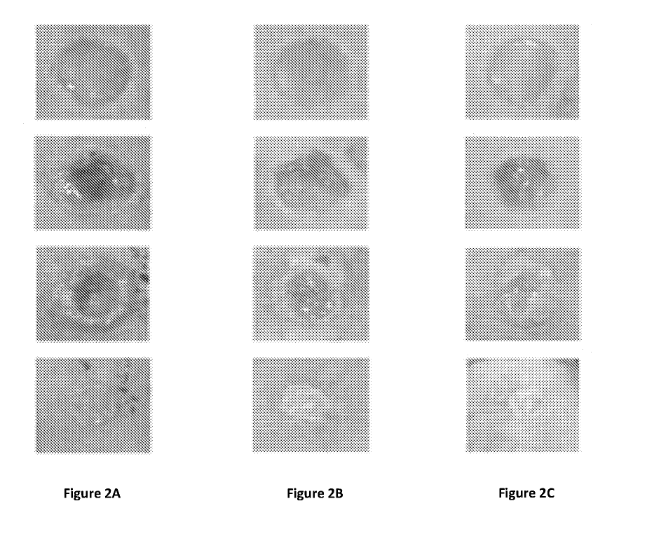

FIG. 2A presents pictures of a wound at different stages of healing thereof, starting from the infliction and up to two weeks from the infliction.

FIGS. 2B and 2C present pictures of wounds at the same stages of healing as the wound in FIG. 2A, wherein the wounds were treated with gauzes prepared from the polymer fiber having 1% wt. of the synergistic combination of copper oxide and tetrasilver tetroxide particles incorporated therein (FIGS. 2B and 2C representing two separate tests). The pictures taken two weeks from the infliction, wherein the wound was treated with the impregnated gauze (bottom pictures of FIGS. 2B and 2C) show wound healing with no scar tissue, therefore demonstrating the cell proliferation properties of the materials of the present invention.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1.-37. (canceled)

38. A material having cell proliferation properties, said material comprising a polymer having incorporated therein a synergistic combination of at least two metal oxide powders, comprising a mixed oxidation state oxide of a first metal and a single oxidation state oxide of a second metal, wherein the mixed oxidation state oxide constitutes from about 25% wt. to about 75% wt. of the total weight of the synergistic combination of the at least two metal oxide powders, the powders are being incorporated substantially uniformly within said polymer, and the ions of the metal oxides are in ionic contact upon exposure of said material to moisture.

39. The material according to claim 38, wherein the mixed oxidation state oxide is selected from the group consisting of tetrasilver tetroxide (Ag₄O₄), Ag₃O₄, Ag₂O₂, tetracopper tetroxide (Cu₄O₄), Cu (I,III) oxide, Cu (II,III) oxide and combinations thereof, and the single oxidation state oxide is selected from the group consisting of copper oxide, silver oxide, zinc oxide and combinations thereof.

40. The material according to claim 39, wherein the synergistic combination of the at least two metal oxide powders comprises copper oxide and tetrasilver tetroxide.

41. The material according to claim 38, wherein each of the mixed oxidation state oxide and the single oxidation state oxide are present in the synergistic combination in a weight percent of about 50%.

42. The material according to claim 38, wherein the at least two metal oxide powders have substantially different specific gravities and substantially similar bulk densities.

43. The material according to claim 42, wherein the metal oxide powders having the substantially similar bulk densities comprise (a) particles which mean particle size is inversely proportional to the specific gravity thereof or (b) particles which have substantially similar mean particles sizes and wherein said particles comprise a coating, wherein the coating thickness is proportional to the specific gravity of the metal oxide particles.

44. The material according to claim 43, wherein the coating comprises polyester or polyalkene wax.

45. The material according to claim 38, wherein the polymer is selected from the group consisting of polyamide, polyester, acrylic, polyalkene, polysiloxane, nitrile, polyvinyl acetate, starch-based polymer, cellulose-based polymer, dispersions and mixtures thereof.

46. The material according to claim 38, wherein the combined weight of the at least two metal oxide powders constitutes from about 0.25% to about 50% wt. of the total weight of the material.

47. The material according to claim 38, being in a form of a master batch, wherein the combined weight of the at least two metal oxide powders constitutes from about 0.5% to about 50% wt. of the total weight of the master batch.

48. The material according to claim 38, being in a form of a fiber, yarn, textile, fabric, film or foil, wherein the combined weight of the at least two metal oxide powders constitutes from about 0.5% to about 15% wt. of the total weight of the material.

49. The material according to claim 48, further comprising a natural fiber selected from the group consisting of cotton, silk, wool, linen and combinations thereof.

50. The material according to claim 38, being in a form of a textile product or a non-textile polymeric article selected from the group consisting of clothing items, bedding textiles, medical textiles including bandages or sutures and textiles for internal and external use.

51. A method for inducing a skin regeneration process, the method comprising applying to the skin of a subject in need of such skin regeneration treatment the material according to claim 38.

52. The method of claim 51, wherein the skin regeneration process is selected from the group consisting of wound healing, accelerated wound closure, and wound healing with reduced scarring.

53. A method for cosmetically improving skin appearance and/or feel, the method comprising applying to the skin of a subject the material according to claim 38.

54. The method of claim 53, wherein the cosmetic improvement comprises an improvement of a condition selected from the group consisting of reduction of wrinkles, reduction of small skin defects, reduction of erythema, reduction of edema, and softening of skin.

55. A method for the preparation of a material having cell-proliferation properties, said material comprising a polymer having incorporated therein a synergistic combination of at least two metal oxide powders comprising a mixed oxidation state oxide of a first metal and a single oxidation state oxide of a second metal, the powders are being incorporated substantially uniformly within said polymer, and are in ionic contact upon exposure of said material to moisture, the method comprising mixing the at least two metal oxide powders with at least one polymer, wherein the mixed oxidation state oxide constitutes from about 25% wt. to about 75% wt. of the total weight of the synergistic combination of the at least two metal oxide powders; and optionally comprising processing the at least two metal oxide powders to have substantially similar bulk densities prior to mixing thereof with the polymer, wherein said processing comprises grinding, applying a coating to the metal oxide powder particles, or both.

56. The method according to claim 55, wherein said mixing comprises producing a master batch, comprising the metal oxide powders and a carrier polymer and adding the master batch to a polymer slurry, wherein said polymer slurry comprises a polymer, which is the same as the carrier polymer or chemically compatible with the carrier polymer.

57. The method according to claim 56, further comprising:
a. forming from the obtained mixture a semi-final product selected from the group consisting of a fiber, yarn, textile, fabric, film and foil, and optionally further comprising blending the obtained fiber with a natural fiber; or
b. forming from the obtained mixture a final product selected from a textile product and a non-textile polymeric article,
said forming performed by a process selected from the group consisting of extrusion, molding, casting and 3D printing.
